(19) United States
(12) Patent Application Publication
(10) Pub. No.: US 2006/0148837 A1
(43) Pub. Date: Jul. 6, 2006

(54) Compositions and methods for treatment of coughing, sneezing, rhinorrhea, and/or nasal obstruction

(75) Inventors: John A. Giordano, West Orange, NJ (US); Rotem Friede, Ardmore, PA (US)

Correspondence Address: PRESTON GATES ELLIS & ROUVELAS MEEDS LLP, 1735 NEW YORK AVENUE, NW, SUITE 500, WASHINGTON, DC, 20006, US

(73) Assignee: Everett Laboratories, Inc.

(21) Appl. No.: 11/028,180

(22) Filed: Jan. 4, 2005

(51) Int. Cl. A61K 31/485; A61K 31/137; A01N 31/14

(52) U.S. Cl. 514/282; 514/649; 514/717; 424/195.18; 514/729

(57) ABSTRACT

The present invention relates to compositions comprising an antitussive, a decongestant and an expectorant, and in a specific embodiment comprising hydrocodone, phenylephrine hydrochloride and guaifenesin, wherein the composition may be substantially free of added sugar and added alcohol, and methods for using these compositions for the treatment of patients suffering from, for example and without limitation, coughing, sneezing, rhinorrhea, and/or nasal obstruction.

### FIELD OF THE INVENTION

The present invention relates to compositions that comprise an antitussive, an expectorant and a decongestant and are free of added sugar and added alcohol, and methods for using these compositions for treatment of patients suffering from, for example and without limitation, coughing, sneezing, rhinorrhea, and/or nasal obstruction.

### BACKGROUND OF THE INVENTION

People suffering from coughing, sneezing, rhinorrhea, and/or nasal obstruction commonly take throat lozenges, cough syrups or cough drops for symptomatic relief. While many such medications presently exist, there is room for improvement in the composition of these medications. For instance, many of these medications contain sugar and alcohol while customers would prefer a medication that did not include these substances. Further, many medications contain a combination or variety of antitussives, expectorants and/or decongestants. While a combination or variety may be acceptable to some patients, others may have restrictions due to allergies or other incompatibilities with certain ingredients. Therefore, there is a need for a coughing, sneezing, rhinorrhea, and/or nasal obstruction medication that is free of added sugar and added alcohol that is also restricted to the inclusion of specific antitussives, expectorants, and decongestants.

### SUMMARY OF THE INVENTION

The present invention provides compositions and methods of using these compositions for the therapeutic treatment of coughing, sneezing, rhinorrhea, and/or nasal obstruction. Specifically, for example, the present invention relates to novel compositions of antitussives, expectorants, and decongestants that can be used to treat coughing, sneezing, rhinorrhea, and/or nasal obstruction caused by a variety of factors.

In one embodiment of the present invention, the compositions may include hydrocodone, phenylephrine hydrochloride and guaifenesin and may be administrable to a patient.

In another embodiment of the present invention, the compositions may be substantially free of added sugar. In another embodiment, the compositions of the present invention may be substantially free of added alcohol. In another embodiment of the present invention, the compositions may be substantially free of added sugar and substantially free of added alcohol.

In another embodiment of the present invention, the compositions may be substantially free of other added active ingredients. For example, in one embodiment, the compositions of the present invention may be substantially free of another added antitussive. Specifically, the compositions may be substantially free of, for example and without limitation, one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

In another embodiment of the present invention, the compositions may be substantially free of other added active ingredients, such as, for example and without limitation, another decongestant. In one embodiment of the present invention, the compositions may be substantially free of any other added nasal decongestant. In another embodiment, the compositions of the present invention may be substantially free of any other added decongestants such as, for example and without limitation, one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

In another embodiment of the present invention, the compositions may be substantially free of other added active ingredients, such as, for example and without limitation, another opioid analgesic. In one embodiment the compositions of the present invention may be substantially free of other added opioid analgesics such as, for example and without limitation, one or more of the group consisting of codeine, morphine, hydromorphone, hydrocodone, oxymorphone, levorphanol, fentanyl, propoxyphene, diphenoxylate, meperidine, methadone, and oxycodone.

In another embodiment of the present invention, the compositions may be substantially free of other added active ingredients, such as, for example and without limitation, another expectorant. In one embodiment the compositions of the present invention may be substantially free of other added expectorants such as, for example and without limitation, one or more of the group consisting of ammonium chloride, ammonium carbonate, acetylcysteine, antimony potassium tartrate, glycerin, potassium iodide, sodium citrate, terpin hydrate, and tolu balsam.

In another embodiment of the present invention, hydrocodone may be included in the compositions of the present invention in the form of hydrocodone bitartrate.

In another embodiment of the present invention, the compositions may be in a liquid form. In another embodiment of the present invention, the compositions may include a flavorant such as, for example and without limitation, a natural flavorant or an artificial flavorant. Flavorants included in the compositions of the present invention may include, for example and without limitation, anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Flavorants included in the compositions of the present invention also may include fruit essence, for example and without limitation, apple essence, pear essence, peach essence, berry essence, wildberry essence, date essence, blueberry essence, kiwi essence, strawberry essence, raspberry essence, cherry essence, plum essence, pineapple essence, and apricot essence. Further, flavorants included in the compositions of the present invention also may include, for example and without limitation, natural mixed berry flavor, citric acid, malic acid, vanilla, vanillin, cocoa, chocolate, and menthol.

In a specific embodiment, the compositions of the present invention may also include a non-sugar sweetening agent. For example, the compositions of the present invention may include, without limitation, saccharin sodium, maltodextrin, aspartame, potassium acesulfame, neohesperidin dihydrochalcone, sucralose, monoammonium glycyrrhizinate, and mixtures thereof.

In one embodiment of the present invention, the composition may include citric acid, adetate disodium, glycerin, methylparaben, propylparaben, propylene glycol, saccharin sodium, sodium citrate, sorbitol, water, FD&C red 40, and artificial cherry flavor.

In another embodiment of the present invention, the compositions may comprise about 1 mg to about 4 mg hydrocodone bitartrate; about 3 mg to about 9 mg phenylephrine hydrochloride; and about 75 mg to about 225 mg guaifenesin.

In another embodiment of the present invention, the compositions may comprise about 2 mg to about 3 mg hydrocodone bitartrate; about 4 mg to about 8 mg phenylephrine hydrochloride; and about 120 mg to about 180 mg guaifenesin.

In another embodiment of the present invention, the compositions may comprise about 2.25 mg to about 2.75 mg hydrocodone bitartrate; about 5 mg to about 7 mg phenylephrine hydrochloride; and about 135 mg to about 165 mg guaifenesin.

In another embodiment of the present invention, the compositions may comprise about 2.5 mg hydrocodone bitartrate; about 6 mg phenylephrine hydrochloride; and about 150 mg guaifenesin.

The present invention also includes methods of administering the compositions of the invention to patients.

In one embodiment of the present invention, the methods may utilize compositions comprising hydrocodone, phenylephrine hydrochloride and guaifenesin.

In another embodiment of the present invention, the methods may utilize compositions that may be substantially free of added sugar. In another embodiment, the methods may utilize compositions that may be substantially free of added alcohol. In another embodiment of the present invention, the methods may utilize compositions that may be substantially free of added sugar and substantially free of added alcohol.

In another embodiment of the present invention, the methods may utilize compositions that may be substantially free of other added active ingredients. For example, in one embodiment, the methods may utilize compositions that may be substantially free of another added antitussive. Specifically, the methods may utilize compositions that may be substantially free of, for example and without limitation, one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

In another embodiment of the present invention, the methods may utilize compositions that may be substantially free of other added active ingredients, such as, for example and without limitation, another decongestant. In one embodiment of the present invention, the methods may utilize compositions that may be substantially free of any other added nasal decongestant. In another embodiment, the methods may utilize compositions that may be substantially free of any other added decongestants such as, for example and without limitation, one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

In another embodiment of the present invention, the methods may utilize compositions that may be substantially free of other added active ingredients, such as, for example and without limitation, another opioid analgesic. In one embodiment of the present invention, the methods may utilize compositions that may be substantially free of other added opioid analgesics such as, for example and without limitation, one or more of the group consisting of codeine, morphine, hydromorphone, hydrocodone, oxymorphone, levorphanol, fentanyl, propoxyphene, diphenoxylate, meperidine, methadone, and oxycodone.

In another embodiment of the present invention, the methods may utilize compositions that may be substantially free of other added active ingredients, such as, for example and without limitation, another expectorant. In another embodiment of the present invention, the methods may utilize compositions that may be substantially free of other added expectorants such as, for example and without limitation, one or more of the group consisting of ammonium chloride, ammonium carbonate, acetylcysteine, antimony potassium tartrate, glycerin, potassium iodide, sodium citrate, terpin hydrate, and tolu balsam.

In another embodiment of the present invention, the methods may utilize compositions comprising hydrocodone in the form of hydrocodone bitartrate.

In another embodiment of the present invention, the methods may utilize compositions that may be in a liquid form. In another embodiment of the present invention, the methods may utilize compositions that may include a flavorant such as, for example and without limitation, a natural flavorant or an artificial flavorant. Flavorants included in the methods of the present invention may include, for example and without limitation, anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Flavorants included in the methods of the present invention also may include fruit essences, for example and without limitation, apple essence, pear essence, peach essence, berry essence, wildberry essence, date essence, blueberry essence, kiwi essence, strawberry essence, raspberry essence, cherry essence, plum essence, pineapple essence, and apricot essence. Further, flavorants included in the methods of the present invention also may include, for example and without limitation, natural mixed berry flavor, citric acid, malic acid, vanilla, vanillin, cocoa, chocolate, and menthol.

In a specific embodiment, the methods of the present invention may utilize compositions that may include a non-sugar sweetening agent. For example, the methods of the present invention may utilize compositions that may include, without limitation, saccharin sodium, maltodextrin, aspartame, potassium acesulfame, neohesperidin dihydrochalcone, sucralose, monoammonium glycyrrhizinate, and mixtures thereof.

In one embodiment of the present invention, the methods may utilize compositions that may include citric acid, adetate disodium, glycerin, methylparaben, propylparaben, propylene glycol, saccharin sodium, sodium citrate, sorbitol, water, FD&C red 40, and artificial cherry flavor.

In another embodiment of the present invention, the methods may utilize compositions that may comprise about 1 mg to about 4 mg hydrocodone bitartrate; about 3 mg to about 9 mg phenylephrine hydrochloride; and about 75 mg to about 225 mg guaifenesin.

In another embodiment of the present invention, the methods may utilize compositions that may comprise about 2 mg to about 3 mg hydrocodone bitartrate; about 4 mg to about 8 mg phenylephrine hydrochloride; and about 120 mg to about 180 mg guaifenesin.

In another embodiment of the present invention, the methods may utilize compositions that may comprise about 2.25 mg to about 2.75 mg hydrocodone bitartrate; about 5 mg to about 7 mg phenylephrine hydrochloride; and about 135 mg to about 165 mg guaifenesin.

In another embodiment of the present invention, the methods may utilize compositions that may comprise about 2.5 mg hydrocodone bitartrate; about 6 mg phenylephrine hydrochloride; and about 150 mg guaifenesin.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a decongestant” is a reference to one or more decongestants and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal.

The term “administrable” defines a composition that is able to be given to a patient. Likewise, “administering” refers to the act of giving a composition to a patient or otherwise making such composition available to a patient.

Through the inclusion of an antitussive, a decongestant and an expectorant, the compositions and methods of the present invention may alleviate symptoms, such as coughing, sneezing, rhinorrhea, and/or nasal obstruction caused by a variety of factors.

Antitussive drugs may act peripherally to inhibit cough by suppressing the responsiveness of one or more vagal sensory receptors that produce cough. Bolser et al., 86(3) J. APPL. PHYSIOL. 1017-1024 (1999); Adcock, RESPIR. MED. 85, Suppl. A 43-46 (1991); Bolser, 9 PULM. PHARMACOL. 357-364 (1997); Kase, 1 TRENDS PHARMACOL. SCI. 237-239 (1980). Antitussive drugs also may act within the central nervous system at the level of the brain stem, where the basic neural circuitry responsible for cough is located. Bolser et al., supra; Korpas & Tomori, COUGH AND OTHER RESPIRATORY REFLEXES, New York, Karger (1979), Shannon, 9 PULM. PHARMACOL. 343-347 (1997); Shannon et al., NEURAL CONTROL OF BREATHING, edited by Miller et al. 215-224 (1996). Specifically, centrally-acting antitussives are thought to inhibit cough by interfering with the central modulation of afferent signals from the periphery, thereby decreasing sensitivity of the cough center located within the medulla to incoming stimuli. www.rxmed.com (last visited Nov. 19, 2004). Even more specifically, a recent model of the basic cough circuitry suggests that the eupneic respiratory pattern and the cough motor pattern are produced by essentially the same neural components. Although this pattern generator normally controls breathing, its behavior is modified to produce cough by excitatory inputs from medullary second-order interneurons mediating pulmonary rapidly and slowly adapting receptor (RAR and SAR, respectively)-afferent information. Shannon, supra; Shannon et al., supra. Centrally active antitussive drugs may act at any level within this system. For example, these drugs could suppress the responsiveness of components of the central pathway for transmitting vagal sensory information (second-order interneurons) and/or have more complex effects on the motor pattern generator for cough. Bolser & DeGennaro, 662 BRAIN RES. 25-30 (1994); Bolser et al., 113 BR. J. PHARMACOL. 1344-1348 (1994); Chou & Wang, 223 J. PHARMACOL. EXP. THER. 249-253 (1975).

Hydrocodone is an opioid analgesic (semisynthetic narcotic) and potent antitussive. It acts centrally to suppress cough and is approximately three to eight times as potent as codeine on a weight for weight basis. It is believed to cause cough suppression by exerting agonist activity primarily at the mu receptor and perhaps through an ultimate effect on serotonin. Ogawa & Kasuya, 290(1) ARCH. INT. PHARMACODYN. THER. 117-127 (November 1987). In a specific embodiment of the compositions and methods of the present invention, hydrocodone may be included in the form of hydrocodone bitartrate (4,5α-epoxy-3-methoxy-17-methylmorphinan-6-one tartrate (1:1) hydrate (2:5)). In another specific embodiment of the compositions and methods of the present invention, hydrocodone may be included in amounts ranging from about 1 mg to about 4 mg. In another specific embodiment of the compositions and methods of the present invention, hydrocodone may be included in amounts ranging from about 2 mg to about 3 mg. In another specific embodiment of the compositions and methods of the present invention, hydrocodone may be included in amounts ranging from about 2.25 mg to about 2.75 mg. In another specific embodiment of the compositions and methods of the present invention, hydrocodone may be included in an amount of about 2.5 mg.

Phenylephrine hydrochloride ((S)-3-hydroxy-α[(methylamino)methyl]benzene-methanol hydrochloride) is a sympathomimetic amine. It acts as an oral nasal decongestant and laryngeal mucous membrane decongestant, with minimal central nervous stimulation, by stimulating alpha-adrenergic receptors to produce pronounced vasoconstriction in the skin, mucous membranes and the mucosa. www.healthdigest.org. (last visited Dec. 6, 2004). In a specific embodiment of the compositions and methods of the present invention, phenylephrine hydrochloride may be included in amounts ranging from about 3 mg to about 9 mg. In another specific embodiment of the compositions and methods of the present invention, phenylephrine hydrochloride may be included in amounts ranging from about 4 mg to about 8 mg. In another specific embodiment of the compositions and methods of the present invention, phenylephrine hydrochloride may be included in amounts ranging from about 5 mg to about 7 mg. In another specific embodiment of the compositions and methods of the present invention, phenylephrine hydrochloride may be included in an amount of about 6 mg.

Viscous secretion exists in the airway of the human body. This secretion has an important role in imparting suitable temperature and humidity to inhaled air. When its amount is moderate, the secretion in the airway is unconsciously swallowed or expelled with the breath, but usually never is expectorated. Thus, any expectoration suggests that there is something extraordinary in the respiratory system. On the other hand, accumulation of this secretion in the airway is liable to cause an infection via the airway. From this point of view, the removal of the secretion is a matter of great significance in the medical treatment of patients who suffer with a disease in the airway.

In order to facilitate expectoration, medicines referred to as “expectorants” have been used. Most expectorants serve to remove the secretion by diluting it through an increase in secretion by the mucosa of the airway, promotion of separation from the mucosa and enhancement of ciliary beat. Guaifenesin (3-(2-methoxypphenoxy)-1,2-propanediol), also known as glyceryl guaiacolate, is an expectorant. It is readily absorbed from the intestinal tract and is thought to enter airway secretions unmetabolized and to have a direct effect either on the mucus secretion itself or the epithelium. Rubin, 116 CHEST 195-200 (1999). For example, guaifenesin is thought to reduce the thickness of mucus and phlegm secretions by increasing the production of fluids in the respiratory tract thus helping to liquefy and thin airway secretions. The increased flow of less viscid secretions promotes ciliary action and further facilitates the removal of airway secretions. Guaifenesin also may inhibit cough peripherally in the airway, by hydrating airway mucus so that it shields the cough receptors from cough-inducing irritants. Dicpinigaitis & Gayle, 124 CHEST 2178-2181 (2003). These peripheral actions of guaifenesin aid in the removal of accumulated secretions from the trachea, bronchi and lungs, thus changing a dry, non-productive cough to a cough that is more productive and less frequent. See, for example, Gibb, 9(2) PULMONARY REVIEWS.COM (2004) (last visited Dec. 6, 2004).

Guaifenesin also may act to suppress cough through an effect in the central nervous system. Rubin, supra. While the exact mechanism of this action of guaifenesin is not known, it is believed that guaifenesin acts centrally by depressing or blocking nerve impulse transmission at the internuncial neuron level of the subcortical areas of the brain, brainstem and spinal cord thus relaxing both the laryngeal and pharyngeal muscles. www.elephantcare.org (last visited Dec. 6, 2004). In a specific embodiment of the compositions and methods of the present invention, guaifenesin may be included in amounts ranging from about 75 mg to about 225 mg. In another specific embodiment of the compositions and methods of the present invention, guaifenesin may be included in amounts ranging from about 120 mg to about 180 mg. In another specific embodiment of the compositions and methods of the present invention, guaifenesin may be included in amounts ranging from about 135 mg to about 165 mg. In another specific embodiment of the compositions and methods of the present invention, guaifenesin may be included in an amount of about 150 mg.

Sugars found in various products are highly undesirable for a number of reasons. For instance, it is known that products, such as medications, containing a high sugar content, more particularly saccharose, fructose and dextrose, may attack the dental enamel as a result of acid being formed in the mouth by certain bacteria if the teeth are not cleaned properly. In addition, many sugar-containing products are unsuitable for diabetics because of their sugar content. Sugar found in manufactured products also may be undesirable due to the increased caloric content caused by the sugar. Because of these factors, there is a progressively increasing world demand for sugar free products, including medications.

Sugar free versions of products may be manufactured using sugar replacements, such as, for example and without limitation, saccharin sodium, maltodextrin, aspartame, potassium acesulfame, neohesperidin dihydrochalcone, sucralose, monoammonium glycyrrhizinate, and mixtures thereof. These “sugar replacements” have the advantage that they do not decompose to form products that attack the dental enamel as a result of the bacterial flora present in the mouth during metabolism, even if the teeth are not cleaned properly. The “sugar replacements” also are suitable for consumption by diabetics and do not add unneeded or unwanted calories to products such as medications. Thus, in a specific embodiment of the compositions and methods of the present invention, the compositions may be free of any added sugar, and instead, may contain a sugar substitute, such as for example and without limitation, one of the sugar replacements described above.

Many cough and decongestant medications also contain alcohol. For various legal, dietary, cultural and even religious reasons, there is interest in the production of alcohol-free medications. For instance, many alcohol-containing medications cannot be used by children because parents may be concerned about the alcohol content, and children may reject the alcohol bite and astringency characteristic of these products. Indeed, most alcohol-containing medications display the language “Keep Away From Children” on their labels. Similarly large numbers of adults reject alcohol based medications for various personal reasons. For instance and without limitation, those recovering from alcohol abuse avoid medications containing alcohol because of the threat that these substances can trigger a negative response. In addition, many institutionalized individuals are not allowed to use alcohol based medications. Therefore, there is a need for medications that are free of any added alcohol. In a specific embodiment of the compositions and methods of the present invention, the compositions may be free of any added alcohol.

The compositions and methods of the present invention may alleviate symptoms, such as coughing, sneezing, rhinorrhea, and/or nasal obstruction, caused by a variety of conditions. For instance, coughing, sneezing, rhinorrhea, and/or nasal obstruction may be caused by, for example and without limitation, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, or a respiratory disease, such as, for example and without limitation, a cold, acute bronchitis, chronic bronchitis, asthmatic bronchitis, bronchiectasis, pneumonia, lung tuberculosis, silicosis, silicotuberculosis, pulmonary cancer, upper respiratory inflammation (caused by, for example and without limitation, pharyngitis, laryngitis, nasal catarrh), asthma, bronchial asthma, infantile asthma, pulmonary emphysema, pneumoconiosis, pulmonary fibrosis, pulmonary silicosis, pulmonary suppuration, pleuritis, tonsillitis, cough hives, or whooping cough. The compositions and methods of the present invention may provide relief from symptoms caused by all of the above.

Typically cough and decongestant preparations are administered as an elixir in a sweetened aromatic solution of alcohol and water. In the present invention, however, syrups and other liquid vehicles may be used. Although water itself may make up the entire carrier, typical cough formulations preferably contain a co-solvent, for example and without limitation, propylene glycol and glycerin, to assist solubilization and incorporation of water insoluble ingredients, flavorants and the like into the composition. Any such ingredients may be included as desired or needed within the compositions and methods of the present invention as long as they are consistent with the objectives herein defined. For example, it is contemplated that when desirable, flavoring, preserving, suspending, thickening and/or emulsifying agents may be included in the compositions and methods of the present invention.

Flavorants that may be used in accordance with the present invention include those known to those skilled in the art. These flavorants may include, for example and without limitation, natural, artificial and synthetic flavor oils and flavoring aromatic and/or oils, oleoresins and extracts derived from plants, leaves, flowers, fruits, and so forth, and combinations thereof. Non-limiting representative flavor oils include anise oil, cinnamon oil, peppermint oil, spearmint oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Also useful flavorants include fruit essences including apple essence, pear essence, peach essence, berry essence, wildberry essence, date essence, blueberry essence, kiwi essence, strawberry essence, raspberry essence, cherry essence, plum essence, pineapple essence, and apricot essence. Other useful flavorants include aldehydes and esters such as benzaldehyde (cherry, almond), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), mixtures thereof and the like. Honey and artificial honey flavor, as well as natural mixed berry flavor, citric acid, malic acid, vanilla, vanillin, cocoa, chocolate, and menthol may also be used in accordance with the present invention.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation was prepared in liquid swallowable form containing the following active ingredients per 5 ml teaspoonful:

| | | |
|---|---|---|
| Hyrdocodone Bitartrate | 2.5 | mg |
| Phenylephrine Hydrochloride | 6 | mg |
| Guaifenesin | 150 | mg |

Other inactive ingredients include: citric acid, adetate disodium, glycerin, methylparaben, propylparaben, propylene glycol, saccharin sodium, sodium citrate, sorbitol, water, FD&C red 40, and artificial cherry flavor.

Example 2

A study is undertaken to evaluate the effectiveness of the compositions of the present invention in the treatment of patients. The objective of the study is to determine whether oral intake of the compositions of the present invention results in an improvement of the symptoms of coughing, sneezing, rhinorrhea, and/or nasal obstruction.

A double-blind, placebo controlled study is conducted over a three-day period. A total of 120 subjects, all presenting for treatment of symptoms of coughing, sneezing, rhinorrhea, and/or nasal obstruction, are chosen for the study. The patients range in age from 8 to 72 years old.

An initial assessment of the symptoms of each patient is conducted when the patients initially present for treatment. The treating physician rates the severity of the symptoms on a 4-point scale (0: absent; 1: mild; 2: moderate; 3: severe). For inclusion in the study, a patient must be rated with a score of two or above for cough and a total score of at least 5 for the sum of the four selected symptoms.

The 120 subjects chosen for the study are separated into two separate groups of 60. The characteristics of the symptoms between the two groups are comparable. The first group is administered a dose of the composition of the present invention every six hours for three days. The second group is administered a placebo medication that is similar in all respects to the administered composition except for the inclusion of the active ingredients, hydrocodone bitartrate, phenylephrine hydrochloride and guaifenesin every six hours for three days. No other medications are taken by the patients during the assessment period.

Patients self-evaluate their symptoms of coughing, sneezing, rhinorrhea, and nasal obstruction using the same 4-point scale (0: absent; 1: mild; 2: moderate; 3: severe) thirty minutes after each dose administration. Patients also note the presence and severity of adverse effects of taking the medication on the 4-point scale. In addition to the initial assessment on day 1, patients are evaluated at the end of day two and day three by the treating physician.

The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at each dose administration, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in all symptoms measured is observed in the treated subjects over the controls upon completion of the study. The patients' self-evaluations demonstrate that those receiving the composition of the present invention rate their symptoms as more improved than those who receive placebo. The evaluations made by the treating physicians also show that patients who receive the composition of the present invention show greater reduction in scores on the 4-point scale as compared to patients receiving placebo. This study demonstrates the efficacy of the composition of the present invention in treating the symptoms of coughing, sneezing, rhinorrhea and nasal obstruction. Regarding potential adverse effects of taking the medication, there are no significant differences between the two therapeutic groups. Thus, this study demonstrates that the administration of the composition of the present invention is effective at treating symptoms of coughing, sneezing, rhinorrhea, and nasal obstruction as well as well-tolerated by the patients.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

1. A composition comprising hydrocodone, phenylephrine hydrochloride and guaifenesin, wherein said composition is administrable to a patient.

2. The composition of claim 1, wherein said composition is substantially free of added sugar.

3. The composition of claim 1, wherein said composition is substantially free of added alcohol.

4. The composition of claim 1, wherein said composition is substantially free of added sugar and substantially free of added alcohol.

5. The composition of claim 1, wherein said composition is substantially free of other added active ingredients.

6. The composition of claim 5, wherein said other added active ingredient is another antitussive.

7. The composition of claim 6, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

8. The composition of claim 5, wherein said other added active ingredient is another decongestant.

9. The composition of claim 8, wherein said another decongestant comprises a nasal decongestant.

10. The composition of claim 9, wherein said another nasal decongestant comprises one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine * hydrochloride, naphazoline hydrochloride, oxymetalozine hydrochloride, tetrahydrozoline hydrochloride, and xylometazoline hydrochloride.

11. The composition of claim 5, wherein said other added active ingredient is another opioid analgesic.

12. The composition of claim 11, wherein said another opioid analgesic comprises one or more of the group consisting of codeine, morphine, hydromorphone, oxymorphone, levorphanol, fentanyl, propoxyphene, diphenoxylate, meperidine, methadone, and oxycodone.

13. The composition of claim 5, wherein said other added active ingredient is another expectorant.

14. The composition of claim 13, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

15. The composition of claim 1, wherein said hydrocodone comprises hydrocodone bitartrate.

16. The composition of claim 1, wherein said composition is in a liquid form.

17. The composition of claim 1, wherein said composition further comprises a flavorant.

18. The composition of claim 17, wherein said flavorant comprises a natural flavorant.

19. The composition of claim 17, wherein said flavorant comprises an artificial flavorant.

20. The composition of claim 17, wherein said flavorant is selected from one or more of the group consisting of anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil.

21. The composition of claim 17, wherein said flavorant is selected from one or more of the group consisting of apple essence, pear essence, peach essence, berry essence, wildberry essence, date essence, blueberry essence, kiwi essence, strawberry essence, raspberry essence, cherry essence, plum essence, pineapple essence, and apricot essence.

22. The composition of claim 17, wherein said flavorant is selected from one or more of the group consisting of natural mixed berry flavor, citric acid, malic acid, vanilla, vanillin, cocoa, chocolate, and menthol.

23. The composition of claim 1, wherein said composition further comprises a non-sugar sweetening agent.

24. The composition of claim 23, wherein said non-sugar sweetening agent is selected from one or more of the group consisting of saccharin sodium, maltodextrin, aspartame, potassium acesulfame, neohesperidin dihydrochalcone, sucralose, and monoammonium glycyrrhizinate.

25. The composition of claim 1, wherein said composition further comprises citric acid, edetate disodium, glycerin, methylparaben, propylparaben, propylene glycol, saccharin sodium, sodium citrate, sorbitol, water, FD&C red 40, and artificial cherry flavor.

26. The composition of claim 15, wherein said composition comprises about 1 mg to about 4 mg hydrocodone bitartrate.

27. The composition of claim 1, wherein said composition comprises about 3 mg to about 9 mg phenylephrine hydrochloride.

28. The composition of claim 1, wherein said composition comprises about 75 mg to about 225 mg guaifenesin.

29. The composition of claim 15, wherein said composition comprises about 2 mg to about 3 mg hydrocodone bitartrate.

30. The composition of claim 1, wherein said composition comprises about 4 mg to about 8 mg phenylephrine hydrochloride.

31. The composition of claim 1, wherein said composition comprises about 120 mg to about 180 mg guaifenesin.

32. The composition of claim 15, wherein said composition comprises about 2.25 mg to about 2.75 mg hydrocodone bitartrate.

33. The composition of claim 1, wherein said composition comprises about 5 mg to about 7 mg phenylephrine hydrochloride.

34. The composition of claim 1, wherein said composition comprises about 135 mg to about 165 mg guaifenesin.

35. The composition of claim 15, wherein said composition comprises about 2.25 mg to about 2.75 mg hydrocodone bitartrate; about 5 mg to about 7 mg phenylephrine hydrochloride; and about 135 mg to about 165 mg guaifenesin.

36. The composition of claim 15, wherein said composition comprises about 2.5 mg hydrocodone bitartrate.

37. The composition of claim 1, wherein said composition comprises about 6 mg phenylephrine hydrochloride.

38. The composition of claim 1, wherein said composition comprises about 150 mg guaifenesin.

39. The composition of claim 15, wherein said composition comprises about 2.5 mg hydrocodone bitartrate; about 6 mg phenylephrine hydrochloride; and about 150 mg guaifenesin.

40. A method comprising administering to a patient a composition comprising hydrocodone, phenylephrine hydrochloride and guaifenesin.

41. The method of claim 40, wherein said composition is substantially free of added sugar.

42. The method of claim 40, wherein said composition is substantially free of added alcohol.

43. The method of claim 40, wherein said composition is substantially free of added sugar and substantially free of added alcohol.

44. The method of claim 40, wherein said composition is substantially free of other added active ingredients.

45. The method of claim 44, wherein said other added active ingredient is another antitussive.

46. The method of claim 45, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

47. The method of claim 44, wherein said other added active ingredient is another decongestant.

48. The method of claim 47, wherein said another decongestant comprises a nasal decongestant.

49. The method of claim 48, wherein said another nasal decongestant comprises one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

50. The method of claim 44, wherein said other added active ingredient is another opioid analgesic.

51. The method of claim 50, wherein said another opioid analgesic comprises one or more of the group consisting of codeine, morphine, hydromorphone, oxymorphone, levorphanol, fentanyl, propoxyphene, diphenoxylate, meperidine, methadone, and oxycodone.

52. The method of claim 44, wherein said other added active ingredient is another expectorant.

53. The method of claim 52, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

54. The method of claim 40, wherein said hydrocodone comprises hydrocodone bitartrate.

55. The method of claim 40, wherein said composition is in a liquid form.

56. The method of claim 40, wherein said composition further comprises a flavorant.

57. The method of claim 56, wherein said flavorant comprises a natural flavorant.

58. The method of claim 56, wherein said flavorant comprises an artificial flavorant.

59. The method of claim 56, wherein said flavorant is selected from one or more of the group consisting of anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil.

60. The method of claim 56, wherein said flavorant is selected from one or more of the group consisting of apple essence, pear essence, peach essence, berry essence, wildberry essence, date essence, blueberry essence, kiwi essence, strawberry essence, raspberry essence, cherry essence, plum essence, pineapple essence, and apricot essence.

61. The method of claim 56, wherein said flavorant is selected from one or more of the group consisting of natural mixed berry flavor, citric acid, malic acid, vanilla, vanillin, cocoa, chocolate, and menthol.

62. The method of claim 40, wherein said composition further comprises a non-sugar sweetening agent.

63. The method of claim 62, wherein said non-sugar sweetening agent is selected from one or more of the group consisting of saccharin sodium, maltodextrin, aspartame, potassium acesulfame, neohesperidin dihydrochalcone, sucralose, monoammonium glycyrrhizinate, and mixtures thereof.

64. The method of claim 40, wherein said composition further comprises citric acid, edetate disodium, glycerin, methylparaben, propylparaben, propylene glycol, saccharin sodium, sodium citrate, sorbitol, water, FD&C red 40, and artificial cherry flavor.

65. The method of claim 54, wherein said composition comprises about 1 mg to about 4 mg hydrocodone bitartrate.

66. The method of claim 40, wherein said composition comprises about 3 mg to about 9 mg phenylephrine hydrochloride.

67. The method of claim 40, wherein said composition comprises about 75 mg to about 225 mg guaifenesin.

68. The method of claim 54, wherein said composition comprises about 2 mg to about 3 mg hydrocodone bitartrate.

69. The method of claim 40, wherein said composition comprises about 4 mg to about 8 mg phenylephrine hydrochloride.

70. The method of claim 40, wherein said composition comprises about 120 mg to about 180 mg guaifenesin.

71. The method of claim 54, wherein said composition comprises about 2.25 mg to about 2.75 mg hydrocodone bitartrate.

72. The method of claim 40, wherein said composition comprises about 5 mg to about 7 mg phenylephrine hydrochloride.

73. The method of claim 40, wherein said composition comprises about 135 mg to about 165 mg guaifenesin.

74. The method of claim 54, wherein said composition comprises about 2.25 mg to about 2.75 mg hydrocodone bitartrate; about 5 mg to about 7 mg phenylephrine hydrochloride; and about 135 mg to about 165 mg guaifenesin.

75. The method of claim 54, wherein said composition comprises about 2.5 mg hydrocodone bitartrate.

76. The method of claim 40, wherein said composition comprises about 6 mg phenylephrine hydrochloride.

77. The method of claim 40, wherein said composition comprises about 150 mg guaifenesin.

78. The method of claim 54, wherein said composition comprises about 2.5 mg hydrocodone bitartrate; about 6 mg phenylephrine hydrochloride; and about 150 mg guaifenesin.
